(19) United States
(12) Patent Application Publication
(10) Pub. No.: US 2013/0185823 A1
(43) Pub. Date: Jul. 18, 2013

(54) MESOPOROUS SILICA NANOPARTICLE-MEDIATED DELIVERY OF DNA INTO ARABIDOPSIS ROOT

(71) Applicant: Academia Sinica, Taipei (TW)

(72) Inventors: Lin Yun Kuang, Taipei (TW); Chia-An Huang, Taichung City (TW); Yue-Le C. Hsing, Taipei (TW); Feng-Peng Chang, Taitung City (TW); Yann Hung, Taipei (TW); Chung-Yuan Mou, Taipei (TW)

(73) Assignee: Academia Sinica, Taipei (TW)

(21) Appl. No.: 13/742,112

(22) Filed: Jan. 15, 2013

Related U.S. Application Data
(60) Provisional application No. 61/587,010, filed on Jan. 16, 2012.

(51) Int. Cl. C12N 15/82

(52) U.S. Cl. CPC C12N 15/8202
USPC 800/278; 435/468; 435/419; 435/317.1; 800/298

(57) ABSTRACT

Transient gene expression is a powerful tool for plant genomics studies. Recently, the use of nanomaterials has drawn great interest. Delivery with mesoporous silica nanoparticles (MSNs) has many advantages. We used surface-functionalized MSNs to deliver and express foreign DNA in Arabidopsis thaliana root cells without the aid of particle bombardment. Gene expression was detected in the epidermis layer and in the more inner cortex and endodermis root tissues. This method is superior to the conventional gene-gun method to deliver DNA, which delivers the gene to the epidermis layer only. Less DNA is needed for the MSN method. Our system is the first use of nanoparticles to deliver DNA to plants with good efficiency and without external aids. MSNs, with multifunctionality and the capability of cargo delivery to plant cells as we demonstrated, provide a versatile system for biomolecule delivery, organelle targeting, and even agriculture, such as improved nutrient uptake.

### CROSS-REFERENCE TO RELATED APPLICATIONS

This application claims priority to U.S. provisional application No. 61/587,010 which was filed on Jan. 16, 2012.

### BACKGROUND OF THE INVENTION

Development of a method for simple and efficient delivery of DNA into plant cells would greatly facilitate plant functional genomics studies. Here we used surface-functionalized mesoporous silica nanoparticles (MSNs) to deliver and express foreign DNA in Arabidopsis thaliana roots without the aid of particle bombardment. Gene expression was detected in the epidermis layer and in the more inner cortex and endodermis root tissues. This method is superior to the conventional gene-gun method to deliver DNA, which delivers the gene to the epidermis layer only. Also less DNA is needed for the MSN method. Our system is the first use of nanoparticles to deliver DNA to plants with good efficiency and without external aids. Furthermore, we observed the polar movement of MSNs in the epidermis layer, which implies that the MSN particles might be transferred in a cell-to-cell fashion.

Transient gene expression is a powerful tool for plant functional genomics studies and can be easily used with dozens of gene candidates. In recent years, the use of nanomaterials in medical and biological fields has drawn great interest. Delivery with mesoporous silica nanoparticles (MSNs) has many advantages for intracellular delivery of drugs, proteins and biogenic molecules¹⁻³. However, in plants, delivery of cargos (such as DNA) by nanoparticles has been difficult because of the barrier of the plant cell wall. Except for the gene-gun bombardment method, the use of nanoparticles as a carrier to deliver DNA into plant tissues has met with little success⁴⁻⁹. Recently, single-walled carbon nanotubes have been used to carry FITC-conjugated single-stranded DNA into cultured tobacco cells', which has suggested the possibility of developing a nano-carrier with special surface properties for intact plant transformation. Although gold-capped MSNs could deliver plasmid DNA into plant cells by gene-gun delivery¹⁰, autonomous delivery of MSN nanocarriers in a culture medium to live plants is desirable.

To explore the potential of MSNs as carriers for plant applications, we synthesized MSNs of about 50 nm through a surfactant (cetyltrimethylammonium bromide, CTAB) templated sol-gel process¹¹ and labeled them with fluorescein isothiocyanate (Bare/F-MSNs, green fluorescence) or rhodamine B isothiocyanate (Bare/R-MSNs, red fluorescence) for tracking. We also functionalized the surfaces of these nanoparticles with N-trimethoxysilylpropyl-, N,N,N-trimethylammonium chloride (TMAPS), 3-aminopropyl-trimethoxysilane (APTMS), or (3-trihydroxysilyl)propylmethylphosphonate (THPMP) to examine the effects of surface-functional groups on the uptake of the nanoparticles by plant cells (FIG. 1a,b and supplementary methods for detailed synthesis). Hereafter the functionalized MSNs are abbreviated TMAPS/Dye-MSNs, APTMS/Dye-MSNs, and THPMP/Dye-MSNs (Dye=F or R), respectively.

Transmission electron microscopy (TEM) images showed the nanoparticles to be about 50 nm and uniform in size (FIG. 1c and FIGS. 6, 7). FIG. 1d is a TEM image of the highly positive-charged TMAPS/F-MSNs revealing the well-ordered hexagonal pore structure. We measured the zeta potential and hydrodynamic size of F-MSN derivatives (FIG. 8) which confirmed the presence of the functionalized F-MSNs. The hydrodynamic sizes were just slightly larger than those measured with TEM, which meant little aggregation of MSNs in solution. The zeta potential and hydrodynamic size of F-MSN derivatives differed in ½ MS¹² and BY-2 culture media¹³, both common medium formulas in plant tissue culture. The zeta potentials spanned +35 to −6 mV in ½ MS medium (pH 5.2) and +19 to −6 mV in BY-2 culture medium (pH 5.7). In ½ MS medium, the zeta potentials of Bare/F-, TMAPS/F-, and APTMS/F-MSNs were highly positive (FIG. 1e). The inter-particle repulsion is strong enough to keep them from aggregating (FIG. 1f). However, the hydrodynamic size of THPMP/F-MSNs was increased likely because the zeta-potential of THPMP/F-MSNs was below a threshold value (charge neutralization by ionic species), thus causing aggregation in solution. Unlike ½ MS, BY-2 culture medium is a highly salted solution and has slightly higher pH, 5.7. In the high salt medium, Bare/F- and THPMP/F-MSNs showed a significant increase in hydrodynamic size because of charge neutralization. This decrease in zeta potential and increase in size with ½ MS and BY-2 culture medium were also observed for the corresponding R-MSNs.

We used tobacco protoplasts with cell walls removed by enzymatic treatments as a model system for uptake of MSNs by plants. Because tobacco protoplasts occasionally show weak green autofluorescence, we used R-MSN derivatives in this study. Protoplasts isolated from tobacco BY-2 cell lines were incubated with various MSN derivatives at 20 μg/ml for 24 h at 26° C. in BY-2 culture media, and then examined by confocal laser scanning microscopy (CLSM). As shown in FIG. 2a and FIG. 9, both the positively charged TMAPS/R- and APTMS/R-MSNs were internalized in 20% of the cells examined (10⁵ cells/ml), whereas no red fluorescence signal was detected with Bare/R-MSNs, THPMP/R-MSNs, or the untreated control (data not shown). Thus, surface properties and hydrodynamic size may play a crucial role in the internalization of nanoparticles by protoplasts. Moreover, the protoplasts remained spherical with each treatment, so MSN treatments had no serious toxic effects in cells.

We next investigated nanoparticle uptake by intact plants. We co-cultured MSNs with tobacco BY-2 suspension cells, lily pollen tubes, onion epidermal cells, and Arabidopsis thaliana (Col-0) roots. Only Arabidopsis roots showed positive results. A. thaliana has a short life cycle and a small genome with known sequences and thus is a good model plant. Arabidopsis roots were cultured with various types of F-MSNs at 24° C. for 24 h in ½ MS media and then examined by CLSM. Green spots from each type of F-MSNs appeared to accumulate inside root cells, although the amounts in each cell differed (FIG. 2b and FIG. 10). To examine the cell toxicity of each type, roots were stained with propidium iodide (PI), a membrane-impermeable dye that identifies dead cells (loss of membrane integrity) by staining nucleic acids¹⁴. The nuclei of various nanoparticle-treated root cells were not labeled with PI (FIG. 2b and FIG. 10); thus MSNs did not have acute toxic effects in Arabidopsis root cells.

We note the uptake of MSNs was invariably in the cells at the root maturation zone (squared area in FIG. 2c). We then performed time-series observation with TMAPS/F-MSNs. Two hours after incubation, root cells showed weak green signals. MSNs were accumulated unevenly in some cells at the maturation zone after 4-h incubation. Then, at 24 h, a polar distribution was obvious; particles gathered at the upper end of a cell (FIG. 2b). Thus, once a large number of MSNs were internalized into Arabidopsis roots, MSNs moved in a polar fashion inside root cells. In the root system, apical-basal polarity is necessary for plant development and growth. The best-studied example of polarity in plants is the regulated transport of the plant hormone auxin, which is regulated by the polar distribution of transport proteins and secretion systems¹⁵. In Arabidopsis roots, the direction of polar movement of TMAPS/F-MSNs is similar to that of auxin in epidermal cells.

Because TMAPS/F-MSNs showed strong positive charge to adsorb the negative charge of DNA, we next explored the use of TMAPS/F-MSNs as vectors for plant transformation. A plasmid harboring a red fluorescence protein (mCherry) gene driven by a constitutively expressed cauliflower mosaic virus 35S promoter was adsorbed by TMAPS/F-MSNs through electrostatic interactions. The binding affinity of pDNA to TMAPS/F-MSNs was assessed by agarose gel electrophoresis assay. When the ratio of pDNA to MSNs (w/w) was ⅕ or less, no free DNA was found in the gel (FIG. 11), which showed that TMAPS/F-MSNs had sufficient capacity to bind pDNA to form stable nanocomplexes. To further characterize the nanocomplexes in ½ MS medium, we measured the hydrodynamic size and zeta potential of pDNA/MSN nanocomplexes of various ratios (w/w). As shown in FIG. 12a, the mean hydrodynamic size of the nanocomplexes varied with pDNA/MSN ratio. When the ratio was high ( 1/25, less MSNs), pDNA-loaded TMAPS/F-MSNs tended to form larger aggregations. With increasing amounts of TMAPS/F-MSNs (ratio< 1/25), the hydrodynamic diameter decreased, from 661 to 157 nm. The surface charge of nanoparticles appeared to be unaffected by the different pDNA/MSN ratios we investigated; they were similar to that of TMAPS/F-MSNs (FIG. 12b). From these results, we chose a pDNA/MSN ratio of 1/100 for transformation investigations.

Arabidopsis roots were treated with pDNA-coated TMAPS/F-MSNs (0.2 μg pDNA; 20 μg TMAPS/F-MSNs) at 24° C. for 48 h in ½ MS medium. Arabidopsis roots expressing mCherry protein (red) were detected by CLSM (FIG. 2d, e), which indicated that the pDNA was transferred into nuclei and the proteins were synthesized and accumulated to a detectable amount. In addition, TMAPS/F-MSNs could also be detected in the gene-expressed cell (green channel in FIG. 2e). At 24 h, we could detect some red fluorescent signals in root epidermis. At 48 h, red fluorescent signals were found in epidermis, cortex, and endodermis. Compared to the standard gene gun delivery, our method required 5 times less pDNA (0.2 vs. 1 μg pDNA per shot), and pDNA expressed in deeper tissues (cortex and endodermis, FIG. 5a) rather than penetrating only superficially into the target tissue (epidermis) with gene-gun delivery.

We examined transformed root cells by TEM. TMAPS/F-MSNs were found in cell walls and cytoplasm and occasionally in plastid and nucleus (FIG. 3a, b, c, d), but not in organelles associated with an endocytic-related network or in endocytic vesicles that just passed through plasma membrane (white arrows in FIG. 3a). Therefore, the nanoparticles did not enter the roots through the endocytic path. To further confirm that the red color was indeed from the mCherry protein, immune-labeled gold particles (12 nm) was employed to label the protein (FIG. 3e, f). The same root cell showed gold particles (red arrows) along with a few MSNs (black arrow in FIG. 3f). Thus, the CLSM and TEM results substantiated that TMAPS/F-MSNs carried pDNA into deep root tissues without external aids and released pDNA to achieve gene expression.

From these results, we suggest using MSNs to deliver DNA or other molecules into plant cells and achieve plant transformation is a versatile system that may be applied to many plant species.

A fundamental question is how were MSNs internalized into root tissues—by physical penetration or a biologically regulated event? Insights into the MSN uptake mechanism may allow us to control the fate of nanoparticles and their cargo in the intracellular environment. To address this question, Arabidopsis plants were subjected to low temperature (4° C., 34 h) and then treated with TMAPS/F-MSNs for another 16 h. In another experiment, the plants were pretreated with cyclohexamide (CHX, an inhibitor of protein synthesis) for 6 h at 24° C., and then treated with TMAPS/F-MSNs for 30 h. Internalization of the nanoparticles occurred with both treatments (FIG. 4). We assessed the physiological state of Arabidopsis roots by the fluorescein diacetate (FDA) method after their exposure to cold (34 h) and CHX (6 h) separately. FDA becomes fluorescent after entering active cells¹⁴. The degree of fluorescence depends on the physiological and metabolic status of the cell. In response to cold or CHX treatment, fluorescence in Arabidopsis roots was weaker than in the un-treated control (FIG. 13), but TMAPS/F-MSNs were still internalized, so its internalization is primarily an energy-independent event, without biological regulation.

TEM and the uptake mode under low temperature and CHX treatments indicated that TMAPS/F-MSNs enter Arabidopsis root cells primarily by a non-biological pathway (scheme B in FIG. 5b). After entering the plasma membrane, TMAPS/F-MSNs may stay in the cytoplasm or enter other organelles such as plastids or nuclei. This uptake route avoiding endocytic vesicles is a particular advantage for cargo-delivery (scheme C in FIG. 5b).

To conclude, we have demonstrated a novel DNA delivery system for Arabidopsis roots based on the independent use of an MSN system, TMAPS/F-MSNs. The MSN vector system is more efficient than the conventional gene-gun method: it delivers DNA to deeper tissues, cortex and endodermis, versus the epidermis only. TEM images of subcellular distribution of TMAPS/F-MSNs and uptake-mode investigations with low-temperature and CHX treatments indicated that TMAPS/F-MSNs entered Arabidopsis root cells directly via a non-bioregulated pathway. MSNs, with multifunctionality and the capability of cargo delivery to plant cells as we demonstrated, may provide a versatile system for biomolecule delivery, organelle targeting, and even improved nutrient uptake^{8,16}. Furthermore, the polar TMAPS/F-MSN movement by cell-to-cell transport being similar to that of the hormone auxin calls for further studies. Understanding the polar transport mechanism of MSNs in plant roots may help reveal some important principles of plant development. Such studies will be important for the use of nanomaterials and nanotechnology in plant research.

### BRIEF DESCRIPTION OF THE DRAWINGS

FIG. 1 Surface-functionalized mesoporous silica nanoparticles (MSNs). a, Schematic representation of a surface-functionalized MSNs and MSN uptake by a plant cell. b, Various surface-functionalized MSNs. c,d, TEM images of TMAPS functionalized FITC-MSNs (TMAPS/F-MSNs). The mean particle size is 43 nm (c), and nano-channels are hexagonally arranged (d). e,f, Zeta potentials (e) and hydrodynamic sizes (f) of the surface-functionalized MSNs in ½ MS (pH 5.2) and BY-2 (pH 5.7) culture media. Data are mean±SD, n=3. Scale bars in c and d are 100 nm and 10 nm, respectively.

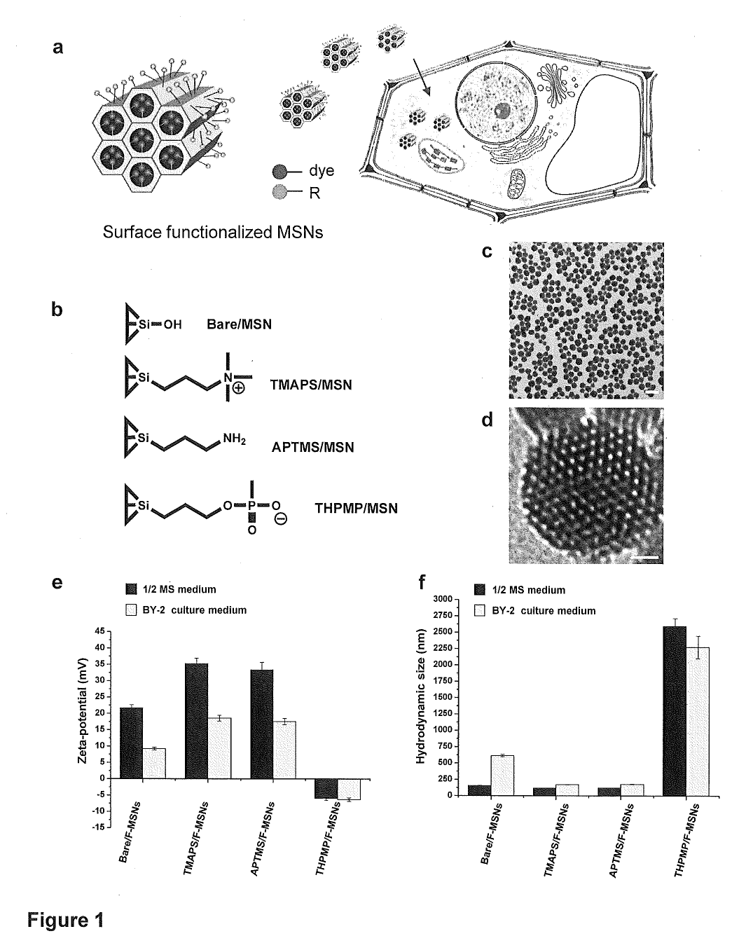

FIG. 2 MSNs enter tobacco protoplasts and Arabidopsis root and deliver cargo into Arabidopsis root. a, Confocal microscopy images of TMAPS/R-MSNs (red; arrows) confirming the uptake by a tobacco BY-2 protoplast. Scale bar: 5 μm. b, Confocal microscopy images of the Arabidopsis root after treatment with TMAPS/F-MSNs (green) for 24 h at 24° C. in ½ MS medium. Roots were stained with PI (red) to label cell walls and reveal cell viability. c, A schematic illustration of the Arabidopsis root. The uptake of TMAPS/F-MSNs was observed in the maturation zone of root as the dashed box in c. Vectorial distribution of TMAPS/F-MSNs in individual cells (arrows) indicated in b and c. d,e, Confocal microscopy images of Arabidopsis root cells treated with pDNA/MSNs (1:100) for 48 h at 24° C. in ½ MS. Gene expression (mCherry, red) was observed in endodermis (d) and cortex (e) cells. TMAPS/F-MSNs were present in the gene-expressed cell (e). Scale bars: 50 μm.

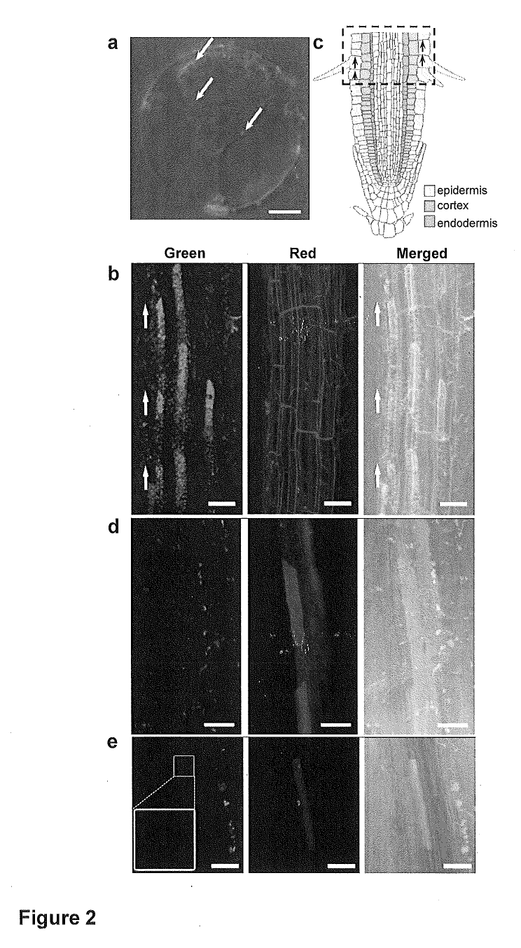

FIG. 3 Subcellular localization of MSN and gene products in Arabidopsis root cell. a,b,c,d, Subcellular localization of TMAPS/F-MSNs by TEM. a, MSNs are present in the cell wall (black arrow) or are passing through the plasma membrane (entered the cell) (white arrows). b, c, d, After penetrating plasma membrane, MSN particles may stay in cytoplasm (b) or enter other organelles such as plastid (c) and nucleus (d) (black arrows). e, f, Immunogold-labelled mCherry protein in root cells after incubation with pDNA-MSN complex. Red arrows are gold-labeled mCherry proteins (e). Co-localization of TMAPS/F-MSNs (black arrow) and mCherry protein (red arrows) in the same cell (f). Scale bars are 200 nm in all panels. Cp, cytoplasm; Cw, cell wall; P, plastid; M, mitochondria; N, nucleus; V, vacuole; G, Golgi apparatus; RER, rough endoplasmic reticulum.

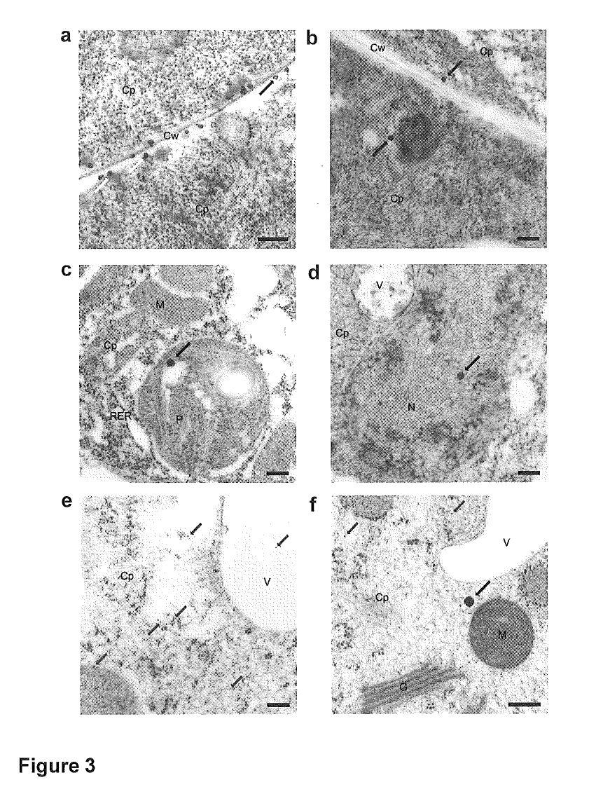

FIG. 4 MSN uptake mechanism in Arabidopsis root. Confocal microscopy images of Arabidopsis roots exposed to a, 4° C. (34 h), then 20 μg TMAPS/F-MSNs at 4° C. for 16 h, and b, 50 μM cyclohexamide (CHX) at 24° C. (6 h), then 20 μg TMAPS/F-MSNs at 24° C. for 30 h. Particles in green (white arrows) were detected inside the root cells. Roots were stained with PI (red) to label cell walls only and reveal cell viability. Scale bars: 20 μm.

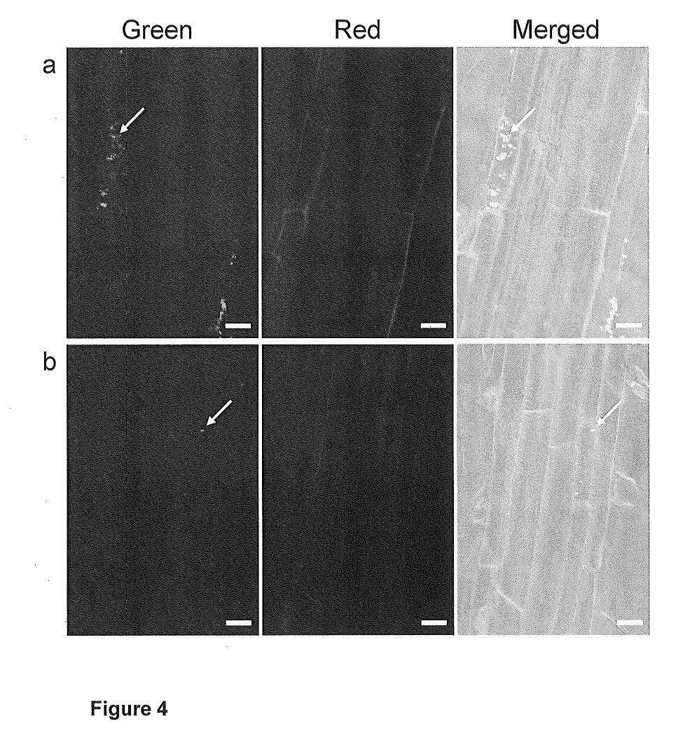

FIG. 5 Summary of MSN internalization into Arabidopsis root. a, Cartoon diagrams of the Arabidopsis plant and cross section of its root. TMAPS/F-MSNs (green) were detected in epidermis and cortex. Gene expression (red) in epidermis, cortex and endodermis indicates DNA molecules delivered to root cells by TMAPS/F-MSNs. b, Possible fate of TMAPS/F-MSNs after internalized into Arabidopsis root cell. Endocytosis is one of the ways to internalize molecules from the extracellular environment (scheme A). TMAPS/F-MSNs may be trapped in the cell wall. After TMAPS/F-MSNs penetrate the plasma membrane, particles could stay in the cytoplasm or enter different organelles such as plastid and nucleus (scheme B). DNA-loaded TMAPS/F-MSN complex internalized into plant cell (scheme C) could approach the nucleus. MSNs and plasmid DNA may pass through the nuclear pore as a complex, or DNA molecules may be released from the MSNs and enter the nucleus.

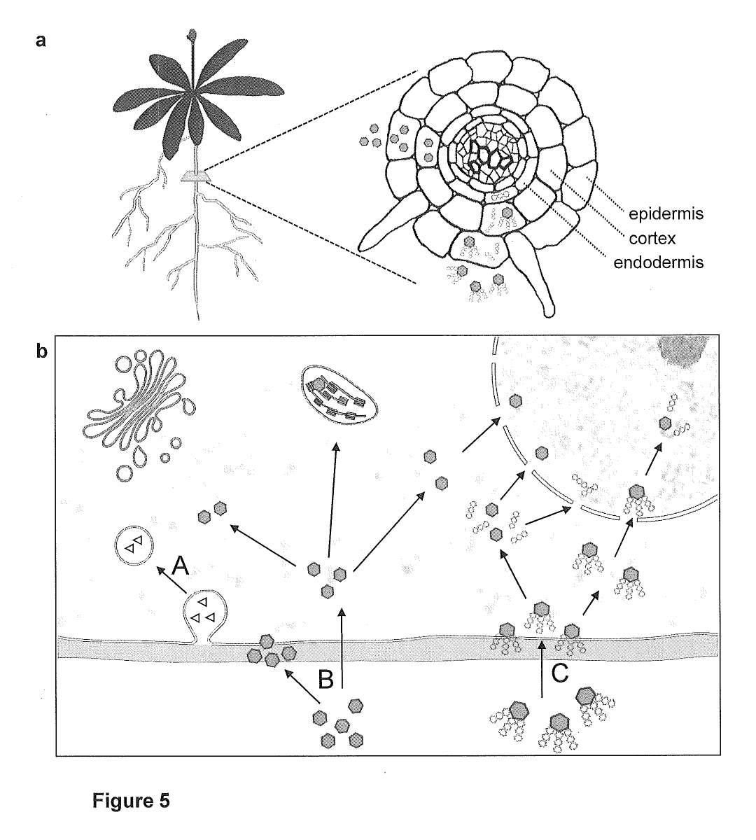

FIG. 6 Transmission electron microscopy (TEM) images of surface-functionalized mesoporous silica nanoparticles (MSNs). a, Bare/F-MSNs, b, APTMS/F-MSNs, and c, THPMP/F-MSNs. Scale bars: 50 nm.

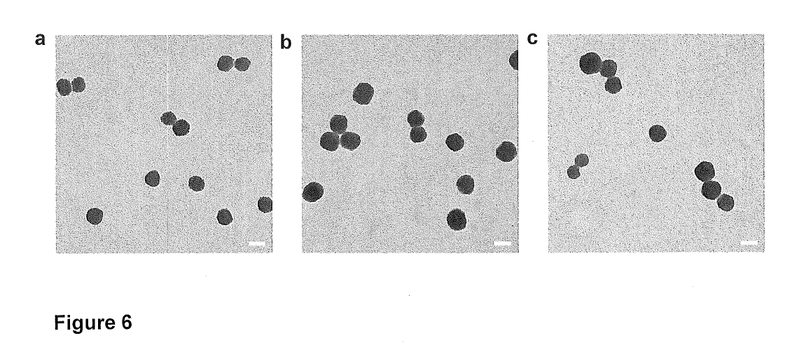

FIG. 7 TEM size distribution of surface-functionalized MSNs. a, Bare/F-MSNs, b, TMAPS/F-MSNs, c, APTMS/F-MSNs, and d, THPMP/F-MSNs.

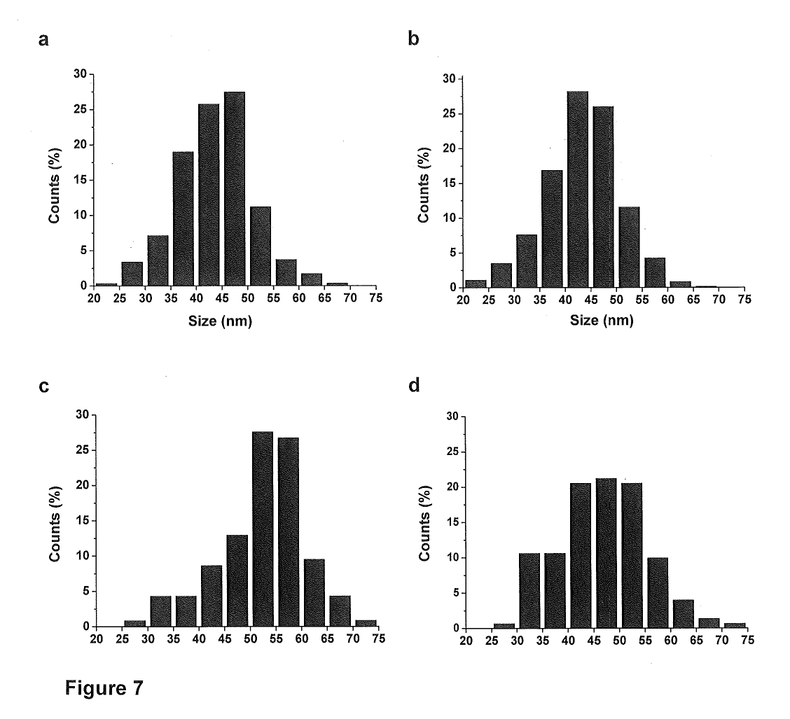

FIG. 8 Zeta potentials and hydrodynamic sizes of surface-functionalized MSNs. a, Zeta values and b, hydrodynamic sizes of surface-functionalized MSNs in aqueous solution. Data are mean±SD, n=3.

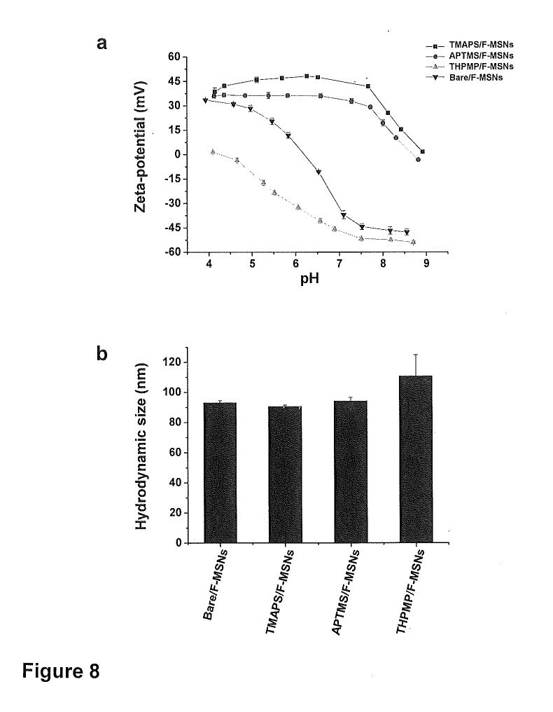

FIG. 9 Confocal microscopy images of APTMS/F-MSN uptake by tobacco BY-2 protoplasts. APTMS/F-MSNs (red) detected in cytoplasm (white arrow). Scale bars: 10 μm.

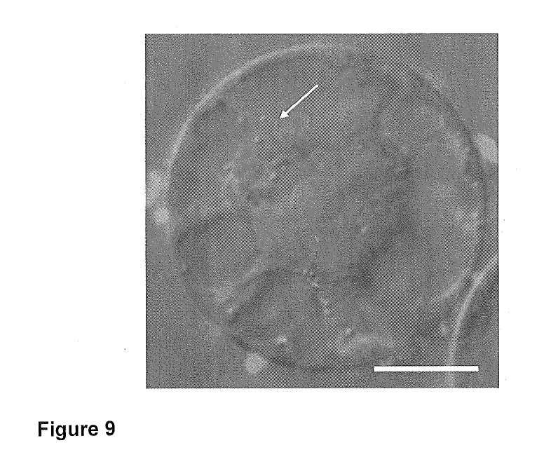

FIG. 10 Confocal microscopy images of surface-functionalized MSN uptake by Arabidopsis roots. Two- to 3-week-old seedlings were cultured in 1 ml ½ MS medium (pH 5.2) containing 20 μg of a, APTMS/F-MSNs, b, Bare/F-MSNs, and c, THPMP/F-MSNs for 24 h at 24° C. After incubation, roots were stained with PI (red) to label cell walls and reveal cell viability. Each type of MSN labeled with FITC (green) is detected inside the root cells (arrows). Scale bars: 50 μm.

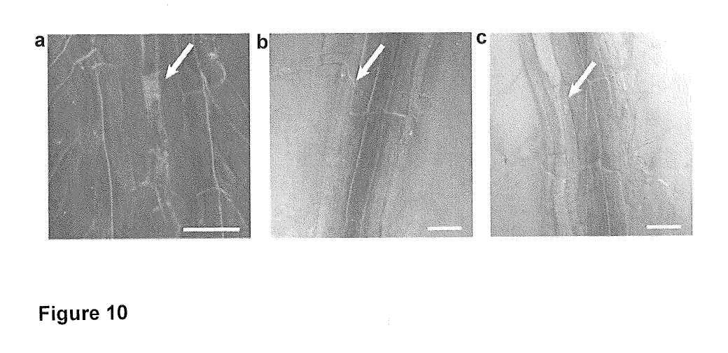

FIG. 11 Agarose gel electrophoresis assay of pDNA-loaded TMAPS/F-MSNs at various DNA/MSN ratios. One microgram DNA (4.5 Kb) was incubated with various amounts of TMAPS/F-MSNs (2, 5, 10, 25, 50, and 75 μg) in ½ MS medium (pH 5.2) for 30 min. The complexes were then electrophorized in 1.5% agarose gel. DNA bands were visualized by ethidium bromide staining. No free DNA bands were observed with >5 μg TMAPS/F-MSNs.

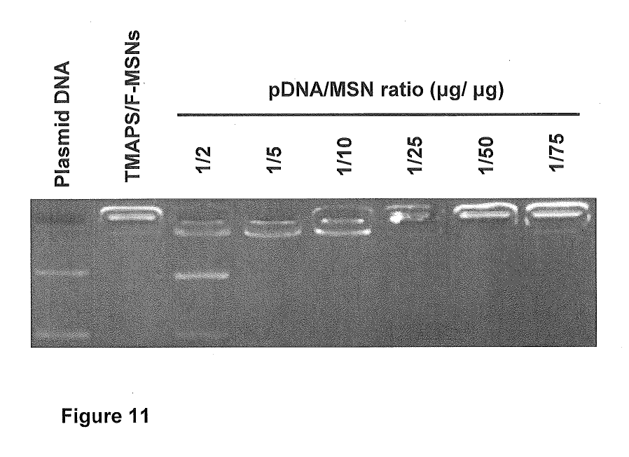

FIG. 12 Characterization of pDNA-loaded TMAPS/F-MSNs by dynamic light-scattering (DLS) and zeta potential measurements. a, Mean particle hydrodynamic size and b, surface charge of the pDNA-loaded TMAPS/F-MSNs at various pDNA/MSN ratios (w/w) in ½ MS medium (pH 5.2). Data are mean±SD, n=3.

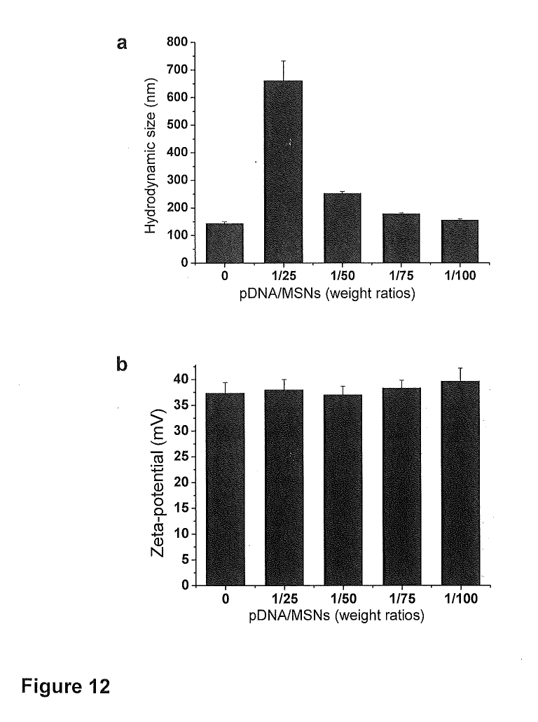

FIG. 13 Assessment of the physiological state of Arabidopsis roots treated at 4° C. or with CHX by FDA stain. Confocal microscopy images of Arabidopsis roots incubated at a, 4° C. and b, 24° C. (control) in ½ MS medium for 34 h; c, with 50 μM CHX and d, without CHX in ½ MS medium at 24° C. for 6 h. Each root was stained with FDA (green) to assess the physiological state. Scale bars: 50 μm. Figures a, c show weak green images, which indicates that the roots were under stress (i.e. weak physiological condition).

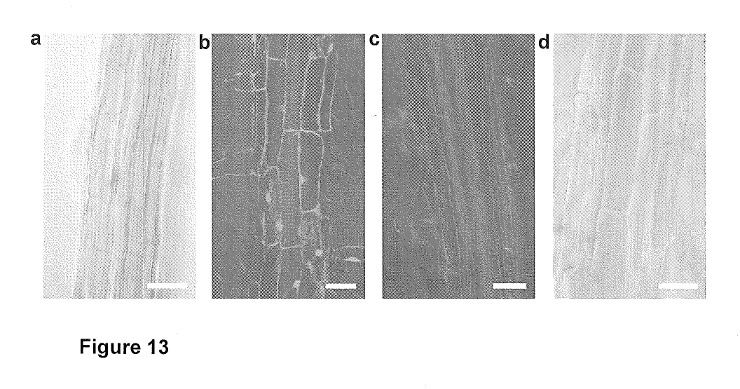

### DETAILED DESCRIPTION OF THE INVENTION

### Methods

### Surface-Functionalized MSN

Fluorescein- or rhodamine-doped MSNs (Bare/F(R)-MSNs) of about 40-50 nm was synthesized as we described¹⁷. The surfactant containing MSNs was functionalized with TMAPS or APTMS by refluxing 2.8 mmole of the corresponding trimethoxysilane with 0.2 g Bare/F(R)-MSNs in ethanol for 12 h. The surfactant templates were then removed as we described¹¹ to obtain TMAPS/F(R)- or APTMS/F(R)-MSNs, respectively. For THPMP modification, the pH of surfactant-containing Bare/F(R)-MSN suspension was adjusted to 10 with NH₄OH (28-30%), and 10 ml of 56 mM aqueous THPMP was added and the mixture was vigorously stirred at 40° C. for 2 h. The surfactant templates were removed to obtain THPMP/F(R)-MSNs.

### Plant Materials

Protoplasts were isolated from Nicotiana tabacum BY-2 suspension cells as described¹³. Arabidopsis seeds (Arabidopsis thaliana Columbia) were surface sterilized with 2% NaOCl containing 0.05% Tween-20 for 15 min, then rinsed thoroughly with sterile water. Surface-sterilized seeds were sown on agar plates containing ½ MS, 3% sucrose (pH 5.8), and 0.8% agar and cultured for 2 to 3 weeks at 24° C. with a 16-h light period.

### MSN Uptake Experiments

For MSN uptake assay, protoplasts were transferred to a new tube, washed twice with W5 solution¹⁵, then diluted to 10⁵ cells/ml with BY-2 culture medium supplemented with 0.4 M mannitol and incubated with various surface-functionalized MSNs at 20 μg/ml. After 24 h, treated cells were washed with BY-2 culture medium, and cellular uptake was analyzed by confocal fluorescence microscopy (Zeiss LSM510). Channel specifications were as follows. FITC-MSNs: excitation, 488 nm, emission, 500-530 nm. RITC-MSNs: excitation, 543 nm, emission, 565-615 nm. mCherry: excitation, 543 nm, emission, 560-615 nm. FDA: excitation, 488 nm, emission, 500-530 nm. PI: excitation, 543 nm, emission, 565-615 nm. For MSN uptake by Arabidopsis roots, 2 to 3-week old seedlings were transferred to ½ MS medium (1 ml; pH 5.2) containing 20 μg of each type of MSNs and incubated for 24 h. After incubation, the roots were washed with ½ MS medium and stained with PI to label cell walls and reveal cell viability. Images were acquired by CLSM.

### DNA-MSN Binding and Plant Transformation

To coat TMAPS/F-MSNs with pDNA for plant transformation, 1 μg of pmCherryl³ was mixed with various amounts of TMAPS/F-MSNs at the ratio of pDNA to MSNs of 1:2, 1:5, 1:10, 1:25, 1:50, and 1:75 in ½ MS medium (pH 5.2). The mixture was immediately vortexed for 5-10 s and then incubated for 30 min at room temperature (RT). Then, the nanocomplex solution was loaded onto 1.5% agarose gel, with naked pDNA as the reference. After gel electrophoresis under 110 V for 60 min, DNA bands were visualized by ethidium bromide staining.

For plant transformation, 2- to 3-week-old Arabidopsis seedlings were transferred to 1 ml ½ MS medium (pH 5.2) containing DNA-TMAPS/F-MSNs (20 μg TMAPS/F-MSNs and 0.2 μg pDNA). After incubation at 24° C. for 48 h, Arabidopsis roots were washed with ½ MS medium (pH 5.2). Gene expression of mCherry protein was observed by CLSM.

### Low Temperature and Cycloheximide (CHX) Experiments

For low-temperature experiments, 2- to 3-week-old Arabidopsis seedlings were cultured in 1 ml pre-cooled medium at 4° C. After 34 h, the roots were stained with fluorescein diacetate (FDA) or further cultured with 20 μg TMAPS/F-MSNs for another 16 h at 4° C. After being washed with ½ MS (pH 5.2) medium, the roots were stained with PI for CLSM assay.

For CHX assay, 2- to 3-week-old Arabidopsis seedlings were pretreated with 50 μM CHX in ½ MS medium for 6 h and then stained with FDA or further treated with 20 μg TMAPS/F-MSNs for another 30 h. After incubation, the roots were washed with ½ MS medium (pH 5.2) and stained with PI for CLSM assay.

### TEM Imaging of Arabidopsis Roots

MSN-internalized roots of Arabidopsis were fixed with 4% paraformaldehyde and 0.1% glutaraldehyde in sodium phosphate buffer, pH 7.0. After 3 rinses with phosphate buffer, the roots were checked and photos were taken by CLSM. Samples were frozen in a high-pressure freezer (Leica EMPACT2) at 2000-2050 bar. Freeze substitution involved anhydrous ethanol with a Leica EM AFS2 (automatic freeze substitution). Samples were kept at −90° C. for 3 days, −60° C. for 1 day, −20° C. for 1 day, 0° C. for 1 day, and then raised to room temperature. The LR White resin was used for infiltration and embedding. Ultrathin sections, 90-120 nm, were cut by use of a Reichert Ultracut S or Lecia EM UC6 (Leica, Vienna, Austria) and collected with 100-mesh nickel grids for TEM.

For immunogold labeling, the individual grids were floated on Tris-buffered saline (TBS) for 15 min, then TBS and 1% bovine serum albumin (BSA) for 15 min. The grids were incubated with primary antibody (Cat. #632543 Clontech, diluted 10× in TBS and 1% BSA) for 1 h. After 4 washes with TBS, the grids were floated on an excess amount (1:20 dilution) of 12 nm colloidal Donkey anti-mouse IgG (Jackson Immuno Research, West Grove, Pa., USA) at room temperature for 1 h, then washed sequentially with 3 droplets of TBS, then ddH₂O for 3 times. After immunogold labeling, the sections were stained with 5% uranyl acetate in water for 10 min and 0.4% lead citrate for 6 min. Sections were observed by TEM (Philips CM 100) at 80 KV, and images were recorded with use of a Gatan Orius CCD camera.

### Supplementary Methods:

### 1. Preparation of MSN

### 1.1. Materials

Ammonium hydroxide (NH₄OH, 28-30 wt %), tetraethyl orthosilicate (TEOS), cetyltrimethylammonium bromide (CTAB), fluorescein isothiocyanate (FITC), and 3-aminopropyltrimethoxysilane (APTMS) were from Acros. (3-trihydroxysilyl)propylmethylphosphonate (THPMP) and N-trimethoxysilylpropyl-N,N,N-trimethylammonium chloride (TMAPS) were from Gelest. Cyclohexamide (CHX), rhodamine B isothiocyanate (RITC), propidium iodide (PI) and fluorescein diacetate (FDA) were from Sigma-Aldrich Chemical. mCherry monoclonal antibody (Cat. #632543) was from Clontech Laboratories. 12 nm Colloidal gold-AffiniPure Donkey anti-mouse IgG was from Jackson Immuno Research. Ultrapure deionized (D.I.) water was generated by a Millipore Milli-Q plus system.

### 1.2. Synthesis of Bare/F-MSNs and Bare/R-MSNs

Dye-functionalized MSNs, RITC-MSNs and FITC-MSNs were prepared by co-condensation. First, N-1-(3-trimethoxysilylpropyl)-N′-fluoresceylthioruea (FITC-APTMS) was formed by stirring FITC ethanolic solution containing APTMS (5 ml of 99.5% ethanol, 1 mg FITC, and 0.56 mmole APTMS) in the dark for 24 h. Separately, 0.58 g CTAB was dissolved in 300 g of 0.17 M NH₄OH at 40° C., and 5 ml of 0.2 M dilute TEOS (in ethanol) was added with stirring. Stirring was continued for 5 h, then 5 ml of FITC-APTMS (in ethanol) and 5 ml of 1.1 M TEOS (in ethanol) was added with vigorous stirring for 1 h. The mixture was then aged at 40° C. for 24 h and centrifuged at 15000 rpm for 30 min. Product was washed with ethanol several times. Finally, surfactant was removed by heating in acidic ethanol (1 g HCl/50 ml ethanol) at 60° C. for 24 h.

Bare/R-MSNs were synthesized by the same procedure, except that RITC was used.

### 1.3. Synthesis of APTMS/F-MSNs, APTMS/R-MSNs, TMAPS/F-MSNs, and TMAPS/R-MSNs

TMAPS and APTMS were grafted onto the external surface of surfactant-containing Bare/Dye-MSNs by refluxing 2.8 mmole of the corresponding trimethoxysilyl derivatives with 0.2 g Bare/Dye-MSNs in ethanol for 12 h. After removing surfactant templates, the desired MSN derivatives were obtained.

### 1.4. Synthesis of THPMP/F-MSNs and THPMP/R-MSNs

For THPMP modification, the pH of surfactant-containing Bare/F-MSN suspension (aged for 22 h in aqueous ammonium) was adjusted to 10 with NH₄OH (28-30%), then 10 ml of 56 mM aqueous THPMP solution was added with vigorous stirring at 40° C. for 2 h. The mixture was centrifuged and washed with ethanol several times. After surfactant was removed by extraction in acidic ethanol, THPMP/F-MSNs were collected. THPMP/R-MSNs were prepared by the same procedure, except surfactant-containing Bare/R-MSN suspension was used.

### 2. Materials Characterization

### 2.1 Zeta-Potential and Dynamic Light-Scattering (DLS) Assays

### 2.11 Surface-Functionalized MSNs in Aqueous Solution

The zeta potentials of surface-functionalized MSNs were characterized in aqueous solution at various pH levels by use of Zetasizer Nano (Malvern; Worcestershire, United Kingdom). Samples were prepared by diluting 3.5 mg of each MSN in 10 ml D.I. water. After ultrasonication for 3 min, solutions were transferred to 1 ml capillary cells, and zeta values were read immediately. The pH value was adjusted with 0.1 N HCl or NaOH by automatic titration. Each zeta value was measured in triplicate.

For DLS assays, 0.35 mg of each surface-functionalized MSN was suspended in 1 ml D.I. water. After ultrasonication for 3 min, hydrodynamic diameters were measured in triplicate.

2.12 Surface-Functionalized MSNs in ½ MS and BY-2 Culture Medium

The pH of ½ MS and BY-2 culture medium was adjusted with 1 N HCl and 1 N NaOH to 5.2 and 5.7, respectively. Samples were prepared by diluting 0.35 mg of each MSN product in 1 ml ½ MS (pH 5.2) or BY-2 culture medium (pH 5.7). After ultrasonication for 3 min, zeta values and the hydrodynamic diameters were measured in triplicate.

### 2.13 DNA/TMAPS-MSN Complexes

To optimize the pDNA/MSN ratios for plant transformation, TMAPS/F-MSNs were incubated with pDNA under diverse pDNA/MSN ratios (1:25, 1:50, 1:75, and 1:100) in 1 ml ½ MS medium (pH 5.2) for 30 min, and the zeta value and hydrodynamic size of each mixture were measured in triplicate by use of a Zetasizer Nano.

### 2.2 TEM Imaging of Surface-Functionalized MSNs

The morphologic features and size of each MSN product were characterized by TEM (Philips CM 100) at 80 KV, and images were recorded by use of a Gatan Orius CCD camera. Ethanolic suspension of samples was dropped onto a carbon-coated copper grid, air dried and examined.

### REFERENCES

1 Trewyn, B. G., Giri, S., Slowing, I. I. & Lin, V. S. Y. Mesoporous silica nanoparticle based controlled release, drug delivery, and biosensor systems. Chemical Communications, 3236-3245 (2007).
2 Wu, S. H., Hung, Y. & Mou, C. Y. Mesoporous silica nanoparticles as nanocarriers. Chemical Communications 47, 9972-9985 (2011).
3 Slowing, I. I., Trewyn, B. G. & Lin, V. S. Y. Mesoporous silica nanoparticles for intracellular delivery of membrane-impermeable proteins. J Am Chem Soc 129, 8845-8849 (2007).
4 Chen, R. et al. Differential Uptake of Carbon Nanoparticles by Plant and Mammalian Cells. Small 6, 612-617 (2010).
5 Corredor, E. et al. Nanoparticle penetration and transport in living pumpkin plants: in situ subcellular identification. Bmc Plant Biol 9, 45 (2009).
6 Lin, S. J. et al. Uptake, Translocation, and Transmission of Carbon Nanomaterials in Rice Plants. Small 5, 1128-1132 (2009).
7 Liu, Q. L. et al. Carbon Nanotubes as Molecular Transporters for Walled Plant Cells. Nano Lett 9, 1007-1010 (2009).
8 Khodakovskaya, M. et al. Carbon Nanotubes Are Able To Penetrate Plant Seed Coat and Dramatically Affect Seed Germination and Plant Growth. Acs Nano 3, 3221-3227, (2009).
9 Kurepa, J. et al. Uptake and Distribution of Ultrasmall Anatase TiO(2) Alizarin Red S Nanoconjugates in Arabidopsis thaliana. Nano Lett 10, 2296-2302, (2010).
10 Torney, F., Trewyn, B. G., Lin, V. S. Y. & Wang, K. Mesoporous silica nanoparticles deliver DNA and chemicals into plants. Nature Nanotechnology 2, 295-300, (2007).
11 Lin, Y. S. et al. Well-ordered mesoporous silica nanoparticles as cell markers. Chemistry of Materials 17, 4570-4573, (2005).
12 Murashige, T. & Skoog, F. A Revised Medium for Rapid Growth and Bio Assays with Tobacco Tissue Cultures. Physiologia Plantarum 15, 473-& (1962).
13 Lee, L. Y., Fang, M. J., Kuang, L. Y. & Gelvin, S. B. Vectors for multi-color bimolecular fluorescence complementation to investigate protein-protein interactions in living plant cells. Plant Methods 4, 24 (2008).
14 Coder, D. M. Assessment of Cell Viability. Current Protocols in Cytometry Ch. 9 (John Wiley & Sons, Inc. Press, New Jersey 1997).
15 Kleine-Vehn, J. & Friml, J. Polar Targeting and Endocytic Recycling in Auxin-Dependent Plant Development. Annu Rev Cell Dev Bi 24, 447-473, (2008).
16 DeRosa, M. C., Monreal, C., Schnitzer, M., Walsh, R. & Sultan, Y. Nanotechnology in fertilizers. Nature Nanotechnology 5, 91-91, (2010).
17 Lu, F., Wu, S. H., Hung, Y. & Mou, C. Y. Size Effect on Cell Uptake in Well-Suspended, Uniform Mesoporous Silica Nanoparticles. Small 5, 1408-1413, (2009).
18 Yoo, S. D., Cho, Y. H. & Sheen, J. Arabidopsis mesophyll protoplasts: a versatile cell system for transient gene expression analysis. Nature Protocols 2, 1565-1572, (2007).

What is claimed:

1. A method of delivering DNA into a plan, the method comprising:
synthesizing surface-functionalized mesoporous silica nanoparticles (MSNs) bound with DNA,
preparing plant materials for uptake of MSNs; and
contacting the MSNs with plant materials for DNA delivery.

2. The method of claim 1, wherein the surface-functionalized MSNs are labeled with a dye for tracking.

3. The method of claim 2, wherein the dye is fluorescein isothiocyanate or rhodamine B isothiocyanate.

4. The method of claim 3, wherein the fluorescein isothiocyanate is Bare/F-MSNs, green fluorescence.

5. The method of claim 3, wherein the rhodamine B isothiocyanate is Bare/R-MSNs, red fluorescence.

6. The method of claim 1, wherein the surface-functionalized MSNs are functionalized with N-trimethoxysilylpropyl-, N, N, N-trimethylammonium chloride (TMAPS), 3-aminopropyl-trimethoxysilane (APTMS), or (3-trihydroxysilyl) propylmethylphosphonate (THPMP).

7. The method of claim 6, wherein the surface-functionalized MSNs are functionalized with N-trimethoxysilylpropyl-,N,N,N-trimethylammonium chloride (TMAPS).

8. The method of claim 6, wherein the surface-functionalized MSNs are functionalized with 3-aminopropyl-trimethoxysilane (APTMS).

9. The method of claim 6, wherein the surface-functionalized MSNs are functionalized with (3-trihydroxysilyl)propylmethylphosphonate (THPMP).

10. The method of claim 1, wherein the plan materials are selected from plan cells, tissues, whole plans, protoplasts, organelles, explants, and plastids.

11. The method of claim 10, wherein the plan materials are protoplasts.

12. The method of claim 11, wherein the plan protoplasts are from tobacco.

13. The method of claim 10, wherein the plan materials are Arabidopsis roots.

14. A transgenic plant cell generated by the method in claim 1.

15. A transgenic plan tissue generated by the method in claim 1.

16. A transgenic plan organelle generated by the method in claim 1.

17. A transgenic plant protoplast generated by the method in claim 1.

18. A transgenic whole plant generated by the method in claim 1.

19. A method of delivering DNA into plan, the method comprising:
synthesizing surface-functionalized mesoporous silica nanoparticles (MSNs) bound with DNA,
labeling the MSNs for tracking;
preparing plant materials for uptake of MSNs; and
contacting the MSNs with plant materials for DNA delivery.

20. A method of delivering DNA into plan, the method comprising:
synthesizing surface-functionalized mesoporous silica nanoparticles (MSNs) bound with DNA,
labeling the MSNs for tracking;
preparing plant materials for uptake of MSNs;
contacting the MSNs with plant materials for DNA delivery; and
detecting the delivered DNA in the plant.

21. The method of claim 1, wherein the labeling for tracking is in the DNA bound with MSNs.
